(19) United States
(12) Patent Application Publication
(10) Pub. No.: US 2023/0302252 A1
(43) Pub. Date: Sep. 28, 2023

(54) NOCTURNAL NEURO DEVICE

(71) Applicants: Joseph B. Arrington, Tooele, UT (US); Lamont D. Bushman, Salt Lake City, UT (US); Shirley L. Ricks, Murray, UT (US); Krischa G. Arrington, Tooele, UT (US); Meghna R. Patel, West Haven, UT (US)

(72) Inventors: Joseph B. Arrington, Tooele, UT (US); Lamont D. Bushman, Salt Lake City, UT (US); Shirley L. Ricks, Murray, UT (US); Krischa G. Arrington, Tooele, UT (US); Meghna R. Patel, West Haven, UT (US)

(21) Appl. No.: 18/126,278

(22) Filed: Mar. 24, 2023

Related U.S. Application Data
(60) Provisional application No. 63/323,467, filed on Mar. 24, 2022.

(51) Int. Cl. A61M 21/00

(52) U.S. Cl. CPC A61M 21/00; A61M 2021/0016; A61M 2021/005; A61M 2021/0022; A61M 2021/0027; A61M 2230/10; A61M 2210/06; A61M 2205/3303

(57) ABSTRACT

While the described nocturnal neuro device can include in any suitable component, in some cases it includes a wearable apparatus for sleep disorder management. Such apparatus can include a housing having a case and a strap. In some cases, the apparatus includes one or more sensors that are configured to monitor one or more physiological indicators of a user, the one or more physiological indicators comprising at least one of a brain electrical activity of the user, a brain wave frequency of the user, and a brain image registered from the user. In some cases, the apparatus further includes a processor to analyze the physiological indicator of the user. In some cases, the apparatus includes a dispensary cartridge for providing olfactory feedback to the user, wherein the dispensary cartridge is configured to dispense an odor from a chemical disposed in the dispensary cartridge. In some cases, when the processor determines that the user is asleep and experiencing a sleep disorder episode, the processor is configured to initiate the olfactory feedback. Other implementations are described.

### CROSS REFERENCE TO RELATED APPLICATION

This application claims priority to U.S. Patent Application No. 63/323,467 (Attorney Docket No. 33975.2), which was filed Mar. 24, 2022, and is entitled NOCTURNAL NEURO DEVICE, the entire disclosure of which is hereby incorporated by reference.

### FIELD

The described systems and methods relate to the treatment of sleep disorders.

### BACKGROUND

A significant number of people suffer from sleep disorders. While people have tried many things to treat such sleep disorders, not all attempts have been successful. Accordingly, there is a need for new additional systems and methods for treating sleep disorders.

### SUMMARY

While the described nocturnal neuro device can include in any suitable component, in some cases it includes a wearable apparatus for sleep disorder management. Such apparatus can include a housing having a case and a strap. In some cases, the apparatus includes one or more sensors that are configured to monitor one or more physiological indicators of a user, the one or more physiological indicators comprising at least one of a brain electrical activity of the user, a brain wave frequency of the user, and a brain image registered from the user. In some cases, the apparatus further includes a processor to analyze the physiological indicator of the user. In some cases, the apparatus includes a dispensary cartridge for providing olfactory feedback to the user, wherein the dispensary cartridge is configured to dispense an odor from a chemical disposed in the dispensary cartridge. In some cases, when the processor determines that the user is asleep and experiencing a sleep disorder episode, the processor is configured to initiate the olfactory feedback.

### BRIEF DESCRIPTION OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a view of a nocturnal neural device being worn on a head of a user in accordance with a representative embodiment;

FIGS. 2-3 each show a schematic view of a neural device being worn on the head of the user in accordance with a representative embodiment;

FIG. 4 illustrates a representative system that provides a suitable operating environment for use with some embodiments of the present invention; and

FIG. 5 illustrates a representative embodiment of a networked system that provides a suitable operating environment for use with some embodiments of the present invention.

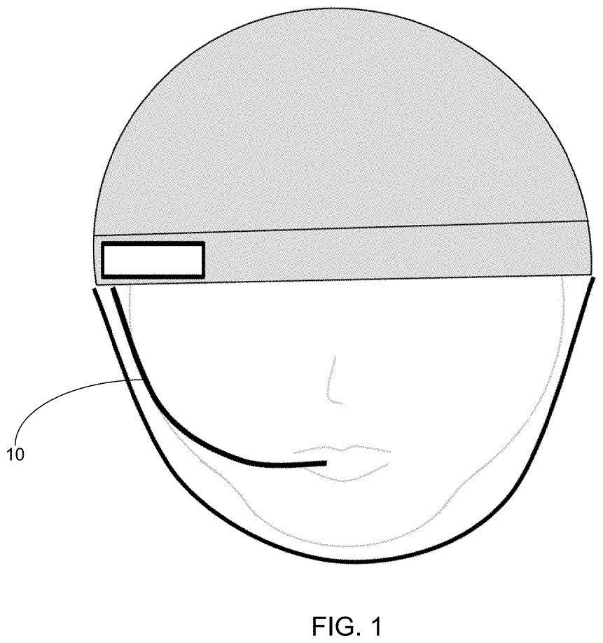

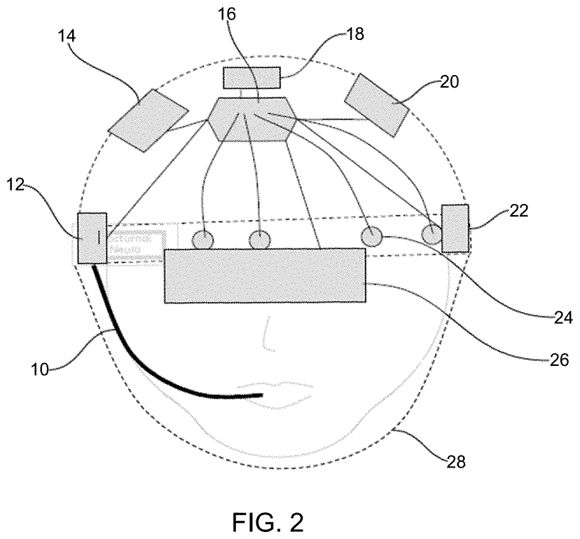

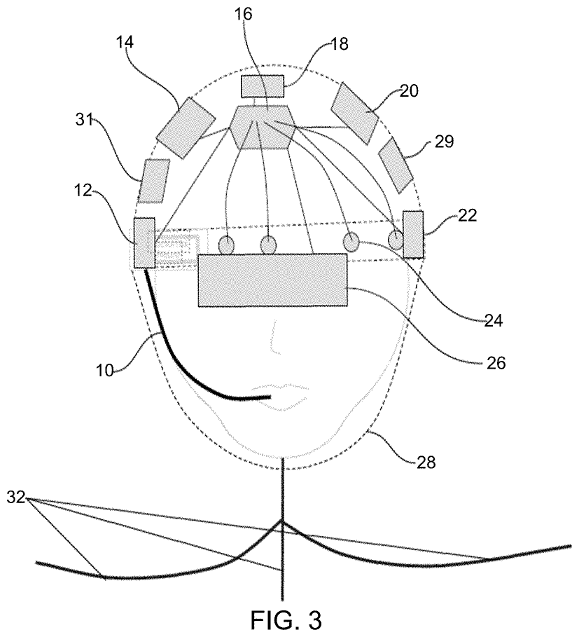

### NOCTURNAL NEURO DEVICE

Sleepwalking, among other sleeping disorders, are labeled as parasomnias, which are abnormal behaviors that occur while one sleeps. Sleepwalking is classified as a potentially dangerous disorder, with victims of the disorder at risk of performing complex tasks such as attempting to handle knives or even driving their car during their sleepwalking episodes! With an estimated overall prevalence of 29% in children and 1.5% in adults, there is a large number of people suffering who need help. Other sleeping disorders, such as restless leg syndrome, sleep paralysis, confusional arousal, night terrors, rapid eye movement (REM) sleep behavior disorder, and many others aggravate quality of life through aggravated sleep. There are millions in the United States alone who suffer from these disorders.

In accordance with some embodiments, the described Nocturnal Neuro Sleep Cap is designed to analyze one or more types of brain waves that are emitted during sleep, picking up on the waves and other physiological indicators dispersed during sleep when sleeping disorders have been shown to be most prevalent. It can be a comfortable cap, headband, sticker based sensor, net or sensors, stocking cap, and/or any other suitable wearable or device that is configured to be placed on the head, with potential extensions running down various portions of the arms and/or back. In accordance with some embodiments, the described sleep cap device is designed so that once a sleep disorder and/or sleep walking event is found, potentially in combination of GPS tracking through a GPS unit on the cap, potentially in combination with an accelerometer to measure movement while user is sleeping, the cap can provide one or more forms of stimuli (in some embodiments, the user can mix and match one or all of them to compound the stimulus): olfactory (the cap would have a slot for a “cannister” or vial of something like a liquid or oil for smells); temperature change; tactile (vibratory); sight (e.g., a screen comes down (and/or visual stimuli is otherwise presented) to provide some sort of visual to help wake, and/or calm waking sleepers, up); auditory (i.e. saved song files, voice messages from loved ones, etc.); gustatory (e.g., one or more small tubes that go into the sleeper's mouth and ejects a small “flavor” onto their tongues to help wake them up). In accordance with some embodiments, the stimuli would continue, increasing in intensity until canceled by the user.

In accordance with some embodiments, the described system can be used to treat sleep walking, in some other embodiments, the system is used to treat a variety of other disorders including sleep paralysis, restless leg, and many others over time as mentioned, but not limited to, earlier descriptions in this body of text). The device's stimuli can be distributed to the user through stimulus giving nodes or other conduits, in some embodiments, that can be over the various nerves running down the back of the user, head of the user, and/or arms of the user. In some embodiments, the system can use brain waves, such as theta, delta, alpha, and gamma but not limited to these, to detect a change in brain activity indicative of a sleep disorder occurring. In some embodiments, the system can use brain imaging to register a sleeping disorder episode occurring. In some embodiments, other brain activity such as magnetic, temperature change, or hormone changes can be used to indicate the beginning of a sleep disorder event occurring.

In accordance with some embodiments, the described system can be used to activate a stimuli based on the registration of a sleep disorder either beginning, or being in the middle of an ongoing episode. In some embodiments, the stimuli can be stacked by the user to be simultaneous. In some embodiments, the stimuli will not cease until the user or other outside person ends the stimuli, thus increasing the strength of the stimuli to ensure the user awakens and becomes conscious, no longer stuck in the episode. In some embodiments, this system can be used to wake users up from nightmares and dreams. In some embodiments, the system can be used to end hallucinations and other imaginary stressors.

In accordance with some embodiments, GPS location can be used to track the user so that if the system detects they are sleeping but moving out of the pre-set location the user is supposed to stay while sleeping, this will cause a stimulus (or stimuli combo). In some embodiments, one or more accelerometers are used to track the movement of the user so that if the user moves but brain activity registers the user is sleeping, it will activate a stimulus or stimuli combo. In some embodiments, both the previously described brain activity and/or GPS tracking and/or accelerometer can be used in conjunction as redundancies for each other to increase the likelihood of catching a sleeping disorder for the user.
10—Straw
12—Gustatory stimuli dispenser
14—Audio stimuli
16—Circuit board
18—Power source
20—Tactile stimuli
22—Olfactory stimuli dispenser
24—EEG sensors
26—Visual stimuli
28—Chinstrap
29—Accelerometer
31—GPS
32—Extensions to arms and back for various other stimuli sensors

### Representative Operating Environment

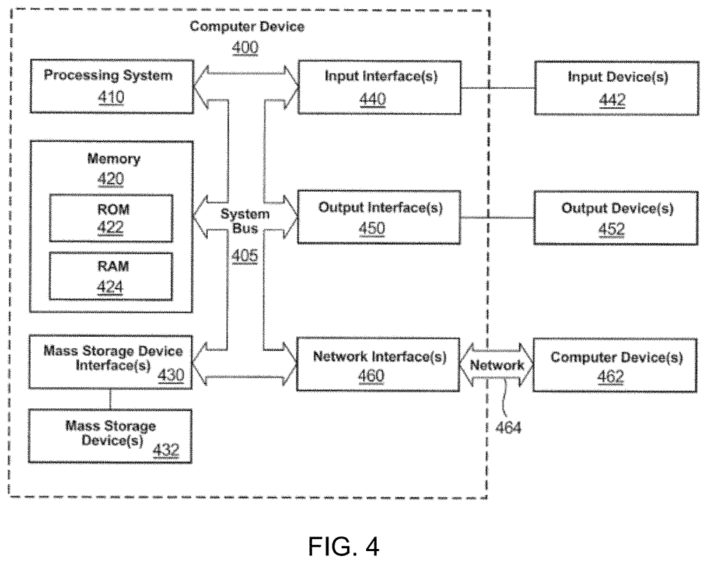

As mentioned previously, some embodiments of the described apparatus comprise one or more processors. In this regard, the described cover cleaner can be used with, or in, any suitable operating environment and/or software. In this regard, FIG. 4 and the corresponding discussion are intended to provide a general description of a suitable operating environment in accordance with some embodiments of the described systems and methods. As will be further discussed below, some embodiments embrace the use of one or more processing (including, without limitation, micro-processing) units in a variety of customizable enterprise configurations, including in a networked configuration, which may also include any suitable cloud-based service, such as a platform as a service or software as a service.

Some embodiments of the described systems and methods embrace one or more computer readable media, wherein each medium may be configured to include or includes thereon data or computer executable instructions for manipulating data. In accordance with some embodiments, the computer executable instructions include data structures, objects, programs, routines, and/or other program modules that can be accessed by one or more processors, such as one associated with a general-purpose processing unit capable of performing various different functions or one associated with a special-purpose processing unit capable of performing a limited number of functions. In this regard, in some embodiments, the processing unit comprises a specialized processing unit that is configured for use with the described cover cleaner 20.

Computer executable instructions cause the one or more processors of the enterprise to perform a particular function or group of functions and are examples of program code means for implementing steps for methods of processing. Furthermore, a particular sequence of the executable instructions provides an example of corresponding acts that may be used to implement such steps.

Examples of computer readable media (including non-transitory computer readable media) include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing unit.

With reference to FIG. 4, a representative system includes computer device 400 (e.g., one or more processors), which may be a general-purpose or special-purpose computer (or processing unit). For example, computer device 400 may be one or more processors, personal computers, notebook computers, PDAs or other hand-held devices, workstations, minicomputers, mainframes, supercomputers, multi-processor systems, network computers, processor-based consumer devices, cellular phones, tablet computers, smart phones, feature phones, smart appliances or devices, control systems, or the like.

Computer device 400 includes system bus 405, which may be configured to connect various components thereof and enables data to be exchanged between two or more components. System bus 405 may include one of a variety of bus structures including a memory bus or memory controller, a peripheral bus, or a local bus that uses any of a variety of bus architectures. Typical components connected by system bus 405 include processing system 410 and memory 420. Other components may include one or more mass storage device interfaces 430, input interfaces 440, output interfaces 450, and/or network interfaces 460, each of which will be discussed below.

Processing system 410 includes one or more processors, such as a central processor and optionally one or more other processors designed to perform a particular function or task. It is typically processing system 410 that executes the instructions provided on computer readable media, such as on the memory 420, a magnetic hard disk, a removable magnetic disk, a magnetic cassette, an optical disk, or from a communication connection, which may also be viewed as a computer readable medium.

Memory 420 includes one or more computer readable media (including, without limitation, non-transitory computer readable media) that may be configured to include or includes thereon data or instructions for manipulating data, and may be accessed by processing system 410 through system bus 405. Memory 420 may include, for example, ROM 422, used to permanently store information, and/or RAM 424, used to temporarily store information. ROM 422 may include a basic input/output system (“BIOS”) having one or more routines that are used to establish communication, such as during start-up of computer device 400. RAM 424 may include one or more program modules, such as one or more operating systems, application programs, and/or program data.

One or more mass storage device interfaces 430 may be used to connect one or more mass storage devices 432 to the system bus 405. The mass storage devices 432 may be incorporated into or may be peripheral to the computer device 400 and allow the computer device 400 to retain large amounts of data. Optionally, one or more of the mass storage devices 432 may be removable from computer device 400. Examples of mass storage devices include hard disk drives, magnetic disk drives, tape drives, solid state mass storage, and optical disk drives.

Examples of solid-state mass storage include flash cards and memory sticks. A mass storage device 432 may read from and/or write to a magnetic hard disk, a removable magnetic disk, a magnetic cassette, an optical disk, or another computer readable medium. Mass storage devices 432 and their corresponding computer readable media provide nonvolatile storage of data and/or executable instructions that may include one or more program modules, such as an operating system, one or more application programs, other program modules, or program data. Such executable instructions are examples of program code means for implementing steps for methods disclosed herein.

One or more input interfaces 440 may be employed to enable a user to enter data (e.g., initial information) and/or instructions to computer device 400 through one or more corresponding input devices 442. Examples of such input devices include a keyboard and/or alternate input devices, such as one or more switches, buttons, dials, sensors (e.g., temperature sensors, G-force sensors, RPM sensors, color sensors, heart rate sensors, blood pressure sensors, conductivity sensors, sweat sensors, and/or any other suitable type of sensors, including, without limitation, those discussed elsewhere herein), digital cameras, pin pads, touch screens, mice, trackballs, light pens, styluses, or other pointing devices, microphones, joysticks, game pads, scanners, camcorders, and/or other input devices. Similarly, examples of input interfaces 440 that may be used to connect the input devices 442 to the system bus 405 include a serial port, a parallel port, a game port, a universal serial bus (“USB”), a firewire (IEEE 1394), a wireless receiver, a video adapter, an audio adapter, a parallel port, a wireless transmitter, or another interface.

One or more output interfaces 450 may be employed to connect one or more corresponding output devices 452 to system bus 405. Examples of output devices include a monitor or display screen, a speaker, a wireless transmitter, a printer, and the like. A particular output device 452 may be integrated with or peripheral to computer device 400. Examples of output interfaces include a video adapter, an audio adapter, a parallel port, and the like.

One or more network interfaces 460 enable computer device 400 to exchange information with one or more local or remote computer devices, illustrated as computer devices 462, via a network 464 that may include one or more hardwired and/or wireless links. Examples of the network interfaces include a network adapter for connection to a local area network (“LAN”) or a modem, BLUETOOTH™, Wi-Fi, a cellular connection, a wireless link, or another adapter for connection to a wide area network (“WAN”), such as the Internet. The network interface 460 may be incorporated with or be peripheral to computer device 400.

3

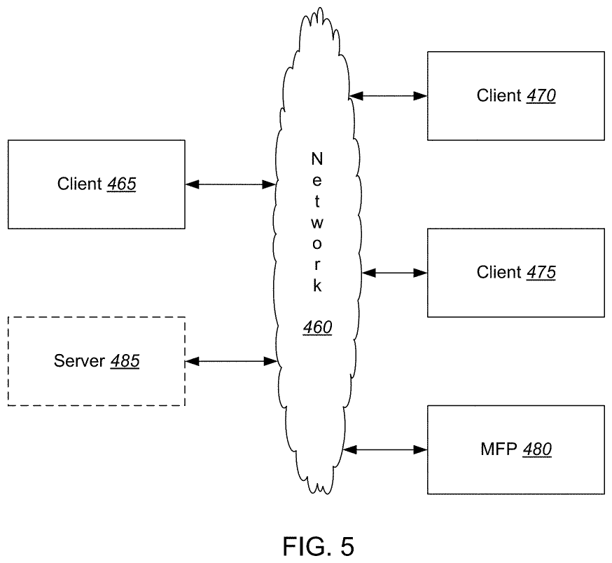

In a networked system, accessible program modules or portions thereof may be stored in a remote memory storage device. Furthermore, in a networked system computer device 400 may participate in a distributed computing environment, where functions or tasks are performed by a plurality networked computer devices. While those skilled in the art will appreciate that the described systems and methods may be practiced in networked computing environments with many types of computer system configurations, FIG. 5 represents an embodiment of a portion of the described systems in a networked environment that includes clients (465, 470, 475, etc.) connected to a server 485 via a network 460. While FIG. 5 illustrates an embodiment that includes 3 clients (e.g., the cover cleaner) connected to the network, alternative embodiments include at least one client connected to a network or many clients connected to a network. Moreover, embodiments in accordance with the described systems and methods also include a multitude of clients throughout the world connected to a network, where the network is a wide area network, such as the Internet. Accordingly, in some embodiments, the described systems and methods can allow for remote: monitoring, training, communication, observation, control, adjustment, troubleshooting, data collecting, system optimization, user interaction, and/or other controlling of the described systems and apparatus from one or more places throughout the world.

What is claimed is:

1. A device comprising a wearable apparatus for sleep disorder management, comprising:
a housing comprising a case and a strap;
a sensor configured to monitor a physiological indicator of a user, the physiological indicator comprising at least one of a brain electrical activity of the user, a brain wave frequency of the user, and a brain image registered from the user;
a processor to analyze the physiological indicator of the user; and
a dispensary cartridge for providing feedback to the user, the dispensary cartridge being configured to be worn on a head of the user when the user wears the device on the head of the user, wherein the dispensary cartridge is configured to provide the feedback, wherein the feedback is selected from at least one of (i) an odor (ii) a gustatory stimulus, (iii) a visual stimulus, (iv) an auditory stimulus, and (v) a tactile stimulus, wherein the processor determines if the user is asleep and experiencing a sleep disorder episode, and wherein the processor is configured to initiate the feedback when the user is asleep and experiencing the sleep disorder episode.

2. (canceled)

3. A device for sleep disorder management, comprising:
a housing comprising a case and a strap;
a sensor configured to monitor a physiological indicator of a user, the physiological indicator comprising at least one of a brain electrical activity of the user, a brain wave frequency of the user, and/or a brain image registered from the user;
a processor to analyze the physiological indicator of the user; and
a screen or other visual image projection for providing a visual feedback to the user, the visual stimulus may be comprising:
a potential screen or image displayer, either to the eyes of the user or onto the surroundings of the user, such portion of the device may protrude from the housing and can be configured to be placed over the head of the user when the user wears the sleep disorder management device on the head of the user that can potentially automatically unroll, pull out, or through other means dispense from the cap to appear in front of the eyes of the use that may play any sort of images, potentially pre-loaded or automated from the device itself, or through other means generated, wherein the processor determines if the user is asleep and experiencing a sleep disorder episode, and wherein the processor is configured to initiate the visual response when the user is asleep and experiencing sleep disorder episode.

4. (canceled)

5. A wearable apparatus for sleep disorder management, comprising:
a housing comprising a case and a strap;
a sensor comprising configured to monitor a physiological indicator of a user, the physiological indicator comprising at least one of a brain electrical activity of the user, a brain wave frequency of the user, and/or a brain image registered from the user;
a processor to analyze the physiological indicator of the user; and
a screen or other visual image projection for providing an auditory feedback to the user, the auditory stimulus may be comprising:
a potential speaker or sound eminator, either to the ears of the user or to the cranium of the user using the audio in a vibratory sense, such portion of the device may protrude from the housing and can be configured to be placed over the head of the user when the user wears the sleep disorder management device on the head of the user that can potentially play any sort of audio, potentially pre-loaded or automated from the device itself, or through other means generated, wherein the processor may determine if the user is asleep and experiencing a sleep disorder episode, and wherein the processor may be configured to initiate the auditory response when the user is asleep and experiencing sleep disorder episode.
